(19) United States
(12) Patent Application Publication
(10) Pub. No.: US 2023/0320771 A1
(43) Pub. Date: Oct. 12, 2023

(54) SURGICAL SCREWDRIVER SYSTEM

(71) Applicant: Spineology Inc., St. Paul, MN (US)

(72) Inventor: Joseph GLEASON, Eagan, MN (US)

(21) Appl. No.: 18/133,514

(22) Filed: Apr. 11, 2023

Related U.S. Application Data
(60) Provisional application No. 63/329,872, filed on Apr. 11, 2022.

(51) Int. Cl. A61B 17/88

(52) U.S. Cl. CPC A61B 17/8875; A61B 17/8897; A61B 2090/033

(57) ABSTRACT

A surgical screwdriver system can include a ratchet handle assembly, a carrier assembly (either left handed or right handed), and a guide pin assembly. A screwdriver forward end can be joined to the handle assembly. A screw can be engaged with the distal end of the screwdriver over a guide pin of the guide pin assembly. A stop knob of the carrier assembly can be adjusted to set the desired depth penetration for the guide pin into the patient's bone or tissue when an impact force, such as from a mallet, is applied to the strike knob of the carrier assembly.

### PRIORITY

This application claims the benefit of U.S. Provisional Application Ser. No. 63/329,872, filed on Apr. 11, 2022, which is hereby incorporated herein by reference in its entirety

### FIELD

The present invention generally relates to screwdriver systems for use in surgical procedures.

### BACKGROUND

Screwdrivers are used in a variety of surgical procedures. For example, a screwdriver can be used to place pedical screws as part of a pedicular fixation system in a spinal surgical procedure. There is a continuing need for screwdrivers for surgical procedures that offer enhanced functionality.

### SUMMARY

The disclosure includes a screwdriver systems, devices and methods that can be used to place a pedicular fixation system on a patient with enhanced convenience and functionality such as described herein and shown/described in the attached drawings. For example, the present screwdriver systems, devices and methods reduce the number of steps, time and complexity of surgical procedures by eliminating separate steps and devices required to place a guide pin or stylet into the patient's tissues and then place a surgical screw into that same tissue.

A surgical screwdriver system can include a ratchet handle assembly, a carrier assembly (either left handed or right handed), and a guide pin assembly. A screwdriver forward end can be joined to the handle assembly. A screw can be engaged with the distal end of the screwdriver over a guide pin of the guide pin assembly. A stop knob of the carrier assembly can be adjusted to set the desired depth penetration for the guide pin into the patient's bone or tissue when an impact force, such as from a mallet, is applied to the strike knob of the carrier assembly.

In another example, a surgical screwdriver system can include a handle and a carrier assembly. The handle can comprise a proximal end, a distal end, and a central bore spanning from the proximal end to the distal end. The carrier assembly can be disposed at least partially within the ratchet handle adjacent to the proximal end. The carrier assembly can comprise a carrier body having a proximal threaded portion and a distal portion, a stop knob threaded onto the proximal threaded portion, and a strike knob secured to a proximal end of the carrier body.

A guide pin can be engaged with the distal portion of the carrier assembly and extend distally from the distal end of the handle. The guide pin can comprise a pin portion and a hub secured to a proximal end of the pin portion.

A screwdriver forward end can be secured to the distal end of the handle. A screw, such as a pedical screw, can be provided to a distal end of the screwdriver forward end. The screw can include a longitudinal bore defined therethrough, and a portion of the guide pin can extend through at least a portion of the longitudinal bore in the screw.

The handle can comprise a ratchet disposed on the distal end thereof. An end cap disposed on the proximal end of the handle. A slide lock can be provided to a proximal end of the handle. For example, the slide lock can be disposed in the end cap and be biased towards a locked position by a spring disposed in the end cap. A dowel pin extending longitudinally from the end cap in a distal direction can also be provided to prevent rotation of the end cap relative to the handle.

The proximal threaded portion of the carrier body can include a plurality of depth dimension markings.

An example of a surgical kit includes a plurality of surgical screws, a guide pin, and a surgical screwdriver. The surgical screwdriver can include a handle and a carrier assembly. The handle can comprise a proximal end, a distal end, and a central bore spanning from the proximal end to the distal end. The carrier assembly can be disposed at least partially within the ratchet handle adjacent to the proximal end. The carrier assembly can comprise a carrier body having a proximal threaded portion and a distal portion, a stop knob threaded onto the proximal threaded portion, and a strike knob secured to a proximal end of the carrier body.

Each screw of the plurality of surgical screws can include a longitudinal bore defined therethrough, which is dimensioned such that a distal portion of the guide pin can extend through at least a portion of the longitudinal bore in the screw. Each screw of the plurality of surgical screws can be a pedical screw for a pedicular fixation system in a spinal surgical procedure.

An example of a method of inserting a screw during a surgical procedure performed on a patient includes placing a screw over a distal end of a surgical screwdriver system such that a guide pin provided to the surgical screwdriver system penetrates through at least a portion of a longitudinal bore defined through the screw; turning a stop knob of a surgical screwdriver system to set a desired depth for a guide pin to penetrate into a bone of the patient; striking a knob on a proximal end of the surgical screwdriver system to cause the guide pin to protrude distally from a distal end of the screw by an amount defined by the desired depth setting step; and rotating a handle of the surgical screwdriver system to insert the screw into the bone of the patient while being guided by the guide wire passing through the bore defined in the screw.

The step of striking the knob on the proximal end of the surgical screwdriver system can include striking a strike knob of the surgical screwdriver system with a mallet. Removing the guide pin from the bone of the patient can include actuating a slide lock and moving the guide pin in a proximal direction.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective view of a screwdriver system in partial transparency according to certain embodiments.

FIG. 2 is an exploded assembly view of the screwdriver system of FIG. 1.

FIG. 3 is a perspective view of a ratcheting handle assembly for a screwdriver system according to certain embodiments.

FIG. 4 is an exploded view of the ratcheting handle assembly of FIG. 3.

FIG. 5 is an exploded view of the carrier assembly and guide pin of the screwdriver system of FIG. 1.

FIG. 6 is a perspective view of the carrier assembly for a screwdriver system according to certain embodiments.

FIG. 7 is an exploded side view of the carrier assembly of FIG. 6 according to certain embodiments.

FIG. 8 is a top view of the carrier assembly of FIG. 6.

FIG. 9 is a side view of a guide pin assembly for a screwdriver system according to certain embodiments.

FIG. 10 is an end view of the guide pin assembly of FIG. 9.

FIG. 11 is a side view of a hub portion indicated in detail B in FIG. 9 according to certain embodiments.

FIG. 12 is an end view of the hub portion indicated in detail B in FIG. 9.

FIG. 13 is a cross-sectional view of the hub portion depicted in FIG. 11 along line A-A of FIG. 10 according to certain embodiments.

FIG. 14 is a side view of a surgical screwdriver system according to certain embodiments.

FIG. 15 is a side view of the screwdriver system of FIG. 14 with a partial cross-section of the ratchet handle assembly according to certain embodiments.

FIG. 16 is a disassembled side view of the screwdriver system of FIG. 14 according to certain embodiments.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

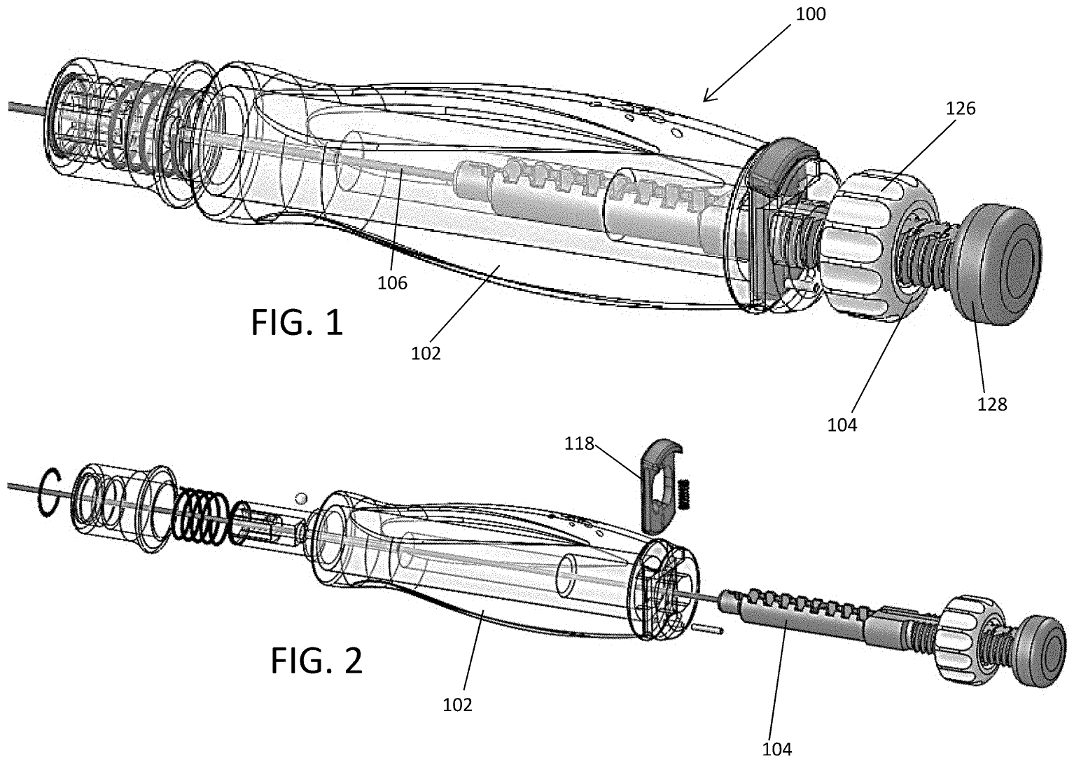

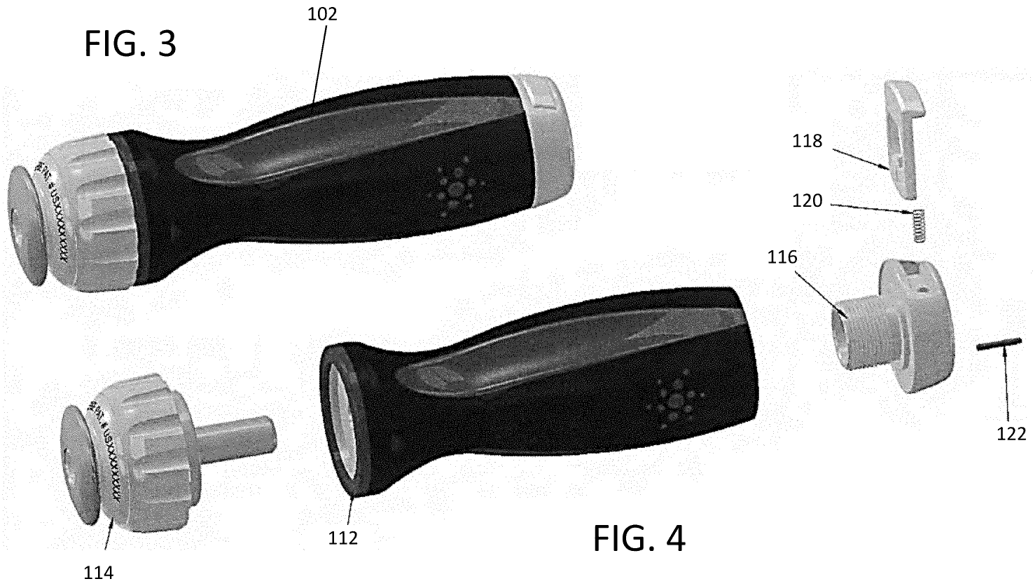

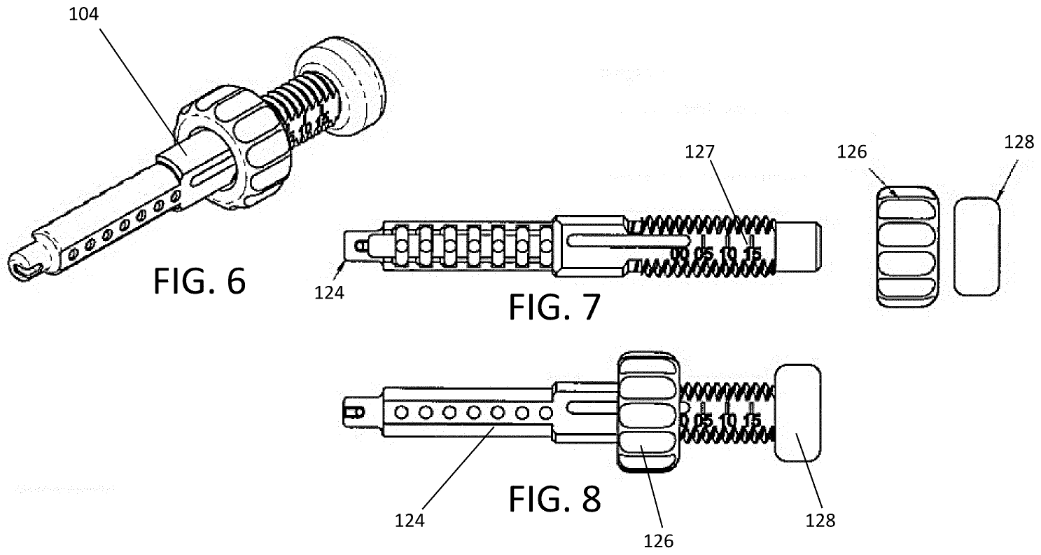

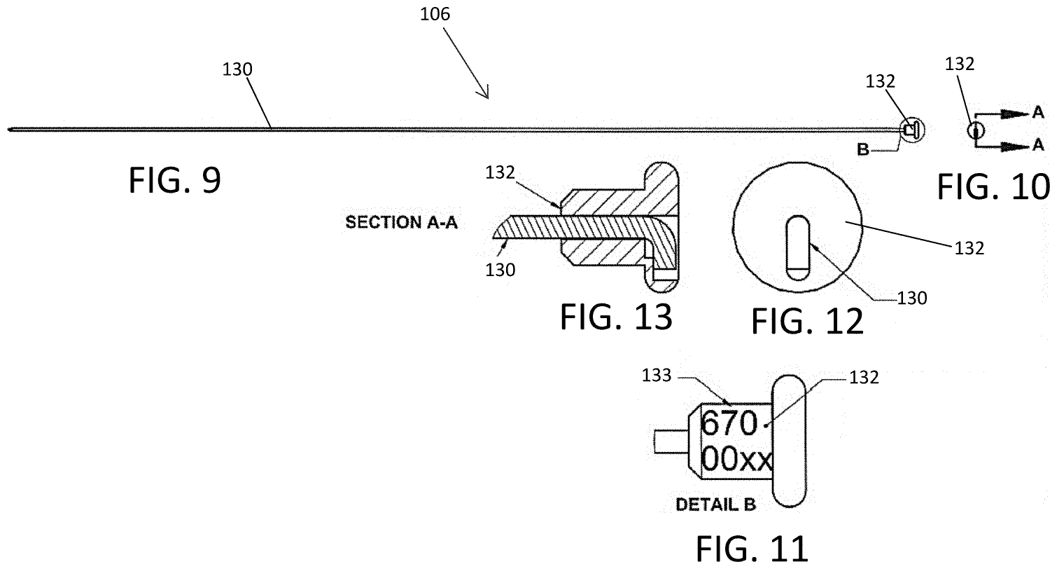

Referring to FIGS. 1-16, a surgical screwdriver system 100 is shown. The screwdriver system 100 includes a ratchet handle assembly 102, a carrier assembly 104 (either left handed or right handed), and a guide pin assembly 106. A screwdriver forward end 108 can be joined to the handle assembly 102. A screw 110 can be engaged with the distal end of the screwdriver 108.

Referring specifically to FIGS. 1-4, the ratchet handle assembly 102 comprises a handle body 112 that includes a longitudinal bore through the center thereof. A ratchet mechanism 114 is joined to a distal end of the handle body 112. An end cap 116 is joined to a proximal end of the handle body 112. The end cap includes a slide lock 118 that is biased via a spring 120 to selectably releasably engage the carrier assembly 104 (as shown in FIGS. 1-2). A dowel pin 122 is also longitudinally provided through the end cap 116 such that it penetrates into the handle body 112 to prevent rotation of the end cap relative to the handle body 112.

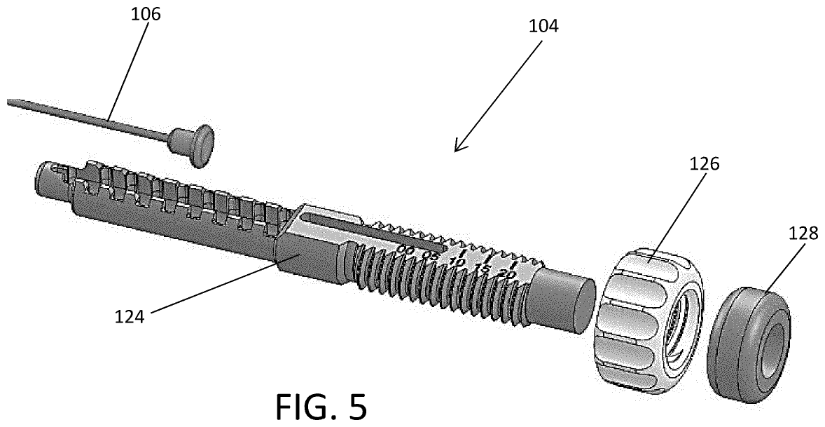

Referring specifically to FIGS. 5-8, the carrier assembly 104 comprises a carrier body 124, a stop knob 126 disposed over a threaded portion of the carrier body 124 and a strike knob 128 secured to the proximal end of the carrier body 124. The carrier body 124 is longitudinally elongated and dimensioned to be inserted at least partially into the proximal end of the handle body 112. A forward or distal portion of the carrier body 124 defines a channel with a series of grooves that are sized to engage a hub of a guide pin or stylet as will be describe below. A rearward or proximal portion of the carrier body 124 is threaded to engage the threads on the inner circumferential surface of the stop knob 126.

A plurality of depth dimension markings 127 are provided on the proximal portion of the carrier body 124 so that the operator or surgeon can see the depth setting that has been selected for penetration of the guide pin into the patient's bone. The strike knob 128 is welded or otherwise suitably secured to the proximal end of the carrier body and presents a flat surface that can be struck by a mallet.

The carrier assembly 104 can be configured for either a left-handed or a right-handed operation. Likewise, the ratcheting mechanism 114 can be configured for either clockwise or counter-clockwise rotation for insertion of the screw.

Referring specifically to FIGS. 5 and 9-13, the guide pin assembly 106 comprises a guide pin 130 or stylet (hereinafter inclusively referred to as the guide pin) with a hub 132 disposed on a proximal end thereof. The hub 132 defines an enlarged or bulbous proximal end portion 134 that is shaped and sized to be secured in one of the grooves defined in the distal portion of the carrier body 124. The proximal end of the guide pin 130 can be secured to the hub 132 by passing through a longitudinal bore in the center of the hub and then bend transversely or perpendicularly to the longitudinal axis, thereby capturing the proximal end of the guide pin 130 in the hub 132. A plurality of identification markings 133 are provided on the hub 132 for quick identification of the guide pin 130 properties by the surgeon.

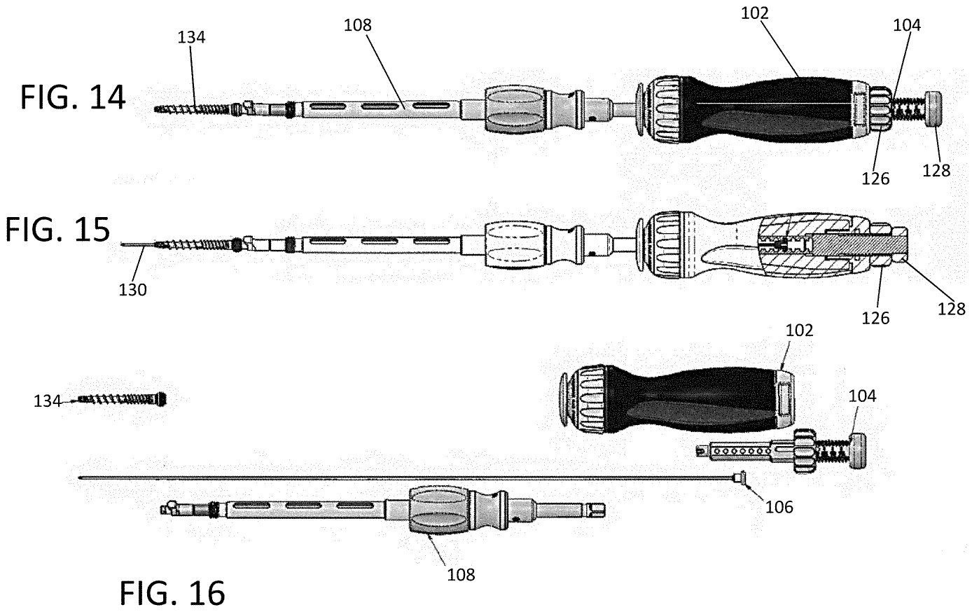

Referring now specifically to FIGS. 14-16, the assembled surgical screwdriver system 100 is shown with a screw 134 engaged with a distal end thereof. The guide pin assembly 106 is engaged with the carrier assembly 104. The carrier assembly 104 is then inserted into the handle assembly 102 and is retained in position via the slide lock 118 snapping into the locked position. The screw 134 is engaged with the distal end of the screwdriver forward end 108 while the distal end portion of the guide pin 134 penetrates through a longitudinal central bore through the screw 134.

The handle assembly 102 is configured to engage the screwdriver forward end 108 via a ¼ turn quick engagement.

In use, the operator or surgeon turns the stop knob 126 to set the desired depth penetration for the guide pin into the patient's bone or tissue. The markings on proximal portion of the carrier body 124 set the depth that the guide pin 130 can extend distally beyond the distal end of the screw 134 when the surgeon hits the strike knob 128 with a mallet. The surgeon contacts the distal end of the screw 134 against the bone or other tissue surface and then hits the strike knob 128 with the mallet. Striking the strike knob 128 by the mallet advances the carrier body 124 relative to the handle body 112 a set distance until the stop knob 126 abuts the proximal end of the carrier handle 112. Thus, the guide pin 130 penetrates the bone or tissue to the pre-set depth. This is shown, for example, in the difference between FIGS. 14 and 15.

The surgeon then turns the handle body 112 in a ratcheting fashion to insert the screw 134 into the patient's bone or tissue. The carrier assembly 104 can be released from the handle assembly 102 by moving the slide lock 118 into the released position.

All of the foregoing assemblies/components can be provided together as part of a surgical kit and housed in a case. The screwdriver system can be assembled, partially assembled or disassembled. A variety of screw diameters/lengths can be included in the kit. Additional components can be included in the kit to perform a particular surgical procedure, such as for example, implanting a pedicular fixation system in a patient.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A surgical screwdriver system, comprising:
a handle, comprising a proximal end, a distal end, and a central bore spanning from the proximal end to the distal end; and
a carrier assembly disposed at least partially within the ratchet handle adjacent to the proximal end; the carrier assembly comprising:
a carrier body, comprising a proximal threaded portion and a distal portion;
a stop knob threaded onto the proximal threaded portion; and
a strike knob secured to a proximal end of the carrier body.

2. The surgical screwdriver system of claim 1, further comprising a guide pin engaged with the distal portion of the carrier assembly and extending distally from the distal end of the handle.

3. The surgical screwdriver system of claim 2, wherein the guide pin comprises a pin portion and a hub secured to a proximal end of the pin portion.

4. The surgical screwdriver system of claim 1, further comprising a screwdriver forward end secured to the distal end of the handle, and a screw provided to a distal end of the screwdriver forward end.

5. The surgical screwdriver system of claim 4, wherein the screw includes a longitudinal bore defined therethrough, and wherein a portion of the guide pin extends through at least a portion of the longitudinal bore in the screw.

6. The surgical screwdriver system of claim 1, wherein the handle comprises ratchet disposed on the distal end thereof.

7. The surgical screwdriver system of claim 6, wherein the handle further comprises an end cap disposed on the proximal end thereof.

8. The surgical screwdriver system of claim 7, wherein the handle further comprises a slide lock provided to a proximal end thereof.

9. The surgical screwdriver system of claim 8, wherein the slide lock is disposed in the end cap and is biased towards a locked position by a spring disposed in the end cap.

10. The surgical screwdriver system of claim 7, wherein the handle further comprises a dowel pin extending longitudinally from the end cap in a distal direction to prevent rotation of the end cap relative to the handle.

11. The surgical screwdriver system of claim 1, wherein the proximal threaded portion of the carrier body includes a plurality of depth dimension markings.

12. A surgical kit, comprising:
a plurality of surgical screws;
a guide pin; and
a surgical screwdriver, the surgical screwdriver comprising:
a handle, comprising a proximal end, a distal end, and a central bore spanning from the proximal end to the distal end; and
a carrier assembly disposed at least partially within the ratchet handle adjacent to the proximal end; the carrier assembly comprising:
a carrier body, comprising a proximal threaded portion and a distal portion;
a stop knob threaded onto the proximal threaded portion; and
a strike knob secured to a proximal end of the carrier body.

13. The kit of claim 12, wherein each screw of the plurality of surgical screws is a pedical screw for a pedicular fixation system in a spinal surgical procedure.

14. The kit of claim 12, wherein each screw of the plurality of surgical screws includes a longitudinal bore defined therethrough, which is dimensioned such that a distal portion of the guide pin can extend through at least a portion of the longitudinal bore in the screw.

15. A method of inserting a screw during a surgical procedure performed on a patient, the method comprising:
placing a screw over a distal end of a surgical screwdriver system such that a guide pin provided to the surgical screwdriver system penetrates through at least a portion of a longitudinal bore defined through the screw;
turning a stop knob of a surgical screwdriver system to set a desired depth for a guide pin to penetrate into a bone of the patient;
striking a knob on a proximal end of the surgical screwdriver system to cause the guide pin to protrude distally from a distal end of the screw by an amount defined by the desired depth setting step; and
rotating a handle of the surgical screwdriver system to insert the screw into the bone of the patient while being guided by the guide wire passing through the bore defined in the screw.

16. The method of claim 15, wherein the step of striking the knob on the proximal end of the surgical screwdriver system includes striking a strike knob of the surgical screwdriver system with a mallet.

17. The method of claim 15, further comprising removing the guide pin from the bone of the patient by actuating a slide lock and moving the guide pin in a proximal direction.
